SEQUENCE LISTING (2 sequences)

SEQ ID NO: 1 - DNA, 49 bp, Artificial
  hCASR_N primer
actaatacgactcactatagggaccatggcattttatagctgctgctgg

SEQ ID NO: 2 - DNA, 29 bp, Artificial
  hCASR_C primer
ttatgaattcactacgttttctgtaacag

(19) United States
(12) Patent Application Publication
(10) Pub. No.: US 2010/0105864 A1
(43) Pub. Date: Apr. 29, 2010

(54) PROPHYLACTIC OR THERAPEUTIC AGENT FOR DIARRHEA

(76) Inventors: Junya Yoneda, Kawasaki-shi (JP); Tetsuo Yano, Kawasaki-shi (JP); Yukie Seki, Kawasaki-shi (JP); Yuzuru Eto, Kawasaki-shi (JP); Yusuke Amino, Kawasaki-shi (JP)

Correspondence Address: CERMAK KENEALY VAIDYA & NAKAJIMA LLP;ACS LLC, 515 EAST BRADDOCK ROAD, SUITE B, ALEXANDRIA, VA, 22314, US

(21) Appl. No.: 12/613,727

(22) Filed: Nov. 6, 2009

Related U.S. Application Data
(63) Continuation of application No. PCT/JP2008/058328, filed on May 1, 2008.

(30) Foreign Application Priority Data
May 8, 2007 (JP) 2007-123765

(51) Int. Cl. C07K 5/08; C07C 211/02; C07K 2/00; C07K 4/00; C07K 5/06

(52) U.S. Cl. 530/331; 564/387; 530/300; 562/400; 562/571

(57) ABSTRACT

A compound which is able to activate a calcium receptor can be used as an active ingredient of a prophylactic or therapeutic agent for treating diarrhea. The compound can be a peptide such as γ-Glu-X-Gly (X represents an amino acid or an amino acid derivative), γ-Glu-Val-Y (Y represents an amino acid or an amino acid derivative), γ-Glu-Ala, γ-Glu-Gly, γ-Glu-Cys, γ-Glu-Met, γ-Glu-Thr, γ-Glu-Val, γ-Glu-Orn, Asp-Gly, Cys-Gly, Cys-Met, Glu-Cys, Gly-Cys, Leu-Asp, γ-Glu-Met(O), γ-Glu-γ-Glu-Val, γ-Glu-Val-NH₂, γ-Glu-Val-ol, γ-Glu-Ser, γ-Glu-Tau, γ-Glu-Cys(S-Me)(O), γ-Glu-Leu, γ-Glu-Ile, γ-Glu-t-Leu, and γ-Glu-Cys(S-Me).

This application is a continuation under 35 U.S.C. §120 of PCT Patent Application No. PCT/JP2008/058328, filed May 1, 2008, which claims priority under 35 U.S.C. §119 to Japanese Patent Application No. 2007-123765, filed on May 8, 2007, which are incorporated in their entireties by reference. The Sequence Listing in electronic format filed herewith is also hereby incorporated by reference in its entirety (File Name: US-418_Seq_List; File Size: 1 KB; Date Created: Nov. 6, 2009).

### BACKGROUND OF THE INVENTION

1. Field of the Invention

The present invention relates to a prophylactic or therapeutic agent for diarrhea, which contains, as an active ingredient, a compound which is able to activate a calcium receptor.

2. Brief Description of the Related Art

The calcium receptor, which is also called the Calcium Sensing Receptor (CaSR), has 1,078 amino acids, and is classified into class C of the seven-transmembrane receptors (G protein-coupled receptor). Cloning of the gene for the calcium receptor was reported in 1993 (Nature, 1993, Vol. 366(6455), pp. 575-580), and the calcium receptor is known to cause various cell responses via elevation of intracellular calcium levels, etc., when activated with calcium etc. The nucleotide sequence of the human calcium receptor is registered with GenBank Accession No. NM_{—}000388, and is well conserved among animals.

The calcium receptor may act to promote or suppress biological functions. Therefore, at present, therapeutic agents are appropriately used in the treatment of diseases of the neurological, hepatic, cardiovascular, and digestive diseases, and other diseases, depending on the pathological conditions. For example, the calcium receptor is able to detect increased blood calcium in the parathyroid, and then suppress the secretion of the parathyroid hormone (PTH) to correct the blood calcium level. Therefore, reduction of the blood calcium level is expected for a calcium receptor activator. It has actually been reported that when a calcium receptor activator is used to treat secondary hyperparathyroidism in a hemodialysis patient, it reduces the PTH level without elevating the calcium and phosphorus levels.

Since a functional analysis of the calcium receptor has been conducted mainly for calcium homeostasis, the applications have so far mainly focused on bone metabolic diseases in which calcium regulation is involved. However, it has become clear from the results of genetic expression analysis, etc., that the calcium receptor is widely distributed in living bodies other than the parathyroid and kidney (J. Endocrinol., 2000, Vol. 165(2), pp. 173-177 and Eur. J. Pharmacol., 2002, Vol. 447(2-3), pp. 271-278), and the possibility that the calcium receptor is involved in various biological functions and perhaps even the causes of some diseases has been proposed. For example, there has been speculation that the calcium receptor is involved in the functions of the liver, heart, lung, gastrointestinal tract, lymphocytes, and pancreas. It has also been confirmed that the calcium receptor is expressed in a wide range of tissues by analyzing RNAs extracted from rat tissues using RT-PCR. Therefore, the potential applications for activators and inhibitors of the calcium receptor are rapidly increasing.

Moreover, in addition to calcium, cations such as a gadolinium cation, basic peptides such as polyarginine, polyamine such as spermine, amino acids such as phenylalanine, and so forth have been reported as calcium receptor activators (Cell Calcium, 2004, Vol. 35(3), pp. 209-216).

It has been reported that glutathione (γ-Glu-Cys-Gly), a low molecular weight peptide, is a calcium receptor activator (J. Biol. Chem., 2006, Vol. 281(13), pp. 8864-8870), but there are no reports of the possibility that glutathione could be effective for the treating diarrhea.

As mentioned above, a number of specific compounds have been developed as calcium receptor activators. However, only a few of these compounds are present in the living body, and further, the compounds that are present in the living body have extremely low activities. Therefore, therapeutic drugs for various diseases that contain these compounds had severe problems in terms of adverse effects, permeability, and sufficient activities. For example, although it was known that an amino acid could act on the calcium receptor, the activity of the amino acid was extremely low. Thus, use of the amino acid to activate the receptor was considered to be difficult. Furthermore, as mentioned above, macromolecules such as polyarginine have been reported as activators, but it is thought that this activity is due to polyvalent cation action of an unspecified structure. In other words, a peptide of a specific structure has not been reported to be useful as a calcium receptor activator.

Diarrhea is a condition that occurs when the moisture present in the stool during defecation is increased, and hence, a loose or liquid stool is excreted. Diarrhea results from the inhibition of moisture absorption due to an intestinal mucosa disorder, the rapid passage of intestine contents due to active peristaltic movement of the intestine, and/or the activation of intestinal juice secretion from the intestinal mucosa, for example.

Diarrhea is classified, based on its mechanism or cause, into six types: osmotic diarrhea; secretory diarrhea; exudative diarrhea; diarrhea associated with an abnormality in intestinal tract motility; diarrhea due to an abnormality in active transport; and others, and the determination of the mechanism or cause of diarrhea is important in the development of diagnostic and therapeutic strategies.

The current therapy for diarrhea caused by a harmful substance, such as a chemical compound, toxin, or infectious bacterium, is to administer an adsorbent, such as kaolin-pectin, which can adsorb the harmful substance. Furthermore, the treatment for diarrhea caused by increased gastrointestinal tract motility is to administer a medicament that acts on the central or peripheral nerves and results in suspension of the gastrointestinal tract motility. Still further, when diarrhea is caused by the invasion of harmful bacteria, an antibiotic or an antimicrobial agent can be administered, provided that the bacterium should be specified.

Although therapeutic drugs have been developed depending on the mechanism or cause of the diarrhea thus far, there are no reports of therapeutic drugs useful to treat diarrhea caused by an electrolyte imbalance in the gastrointestinal tract. A therapeutic drug for diarrhea based on the physiological function inherent to the gastro intestinal tract can be a novel potent therapeutic drug in terms of a function and safety. Therefore, a safe therapeutic drug for diarrhea can be provided.

The authors of Geilbel et al. report the possibility that a calcium receptor activator may serve as a therapeutic drug for diarrhea, but does not disclose whether the calcium receptor actually has a prophylactic or therapeutic effect (Proc. Natl. Acad. Sci. USA, 2006, Vol. 103(25), pp. 9390-9397). Geilbel et al. also mentions that the calcium receptor activator is desirably non-absorbed in the body for safety reasons, but does not elucidate the structure of the compound.

### SUMMARY OF THE INVENTION

It is as aspect of the present invention is to provide a prophylactic or therapeutic agent for diarrhea, which is highly safe in the living body.

Peptides that are able to activate a calcium receptor are described. A compound that is able to activate a calcium receptor can be a therapeutic drug for diarrhea. It is an aspect of the present invention to provide a prophylactic or therapeutic agent for diarrhea, including a compound having a calcium receptor-activating action.

It is another aspect of the present invention to provide the prophylactic or therapeutic agent as described above, in which the compound is selected from the group consisting of a peptide, a peptide derivative, cinacalcet, a compound having the structure of formula (1), and a compound having the structure of formula (2)

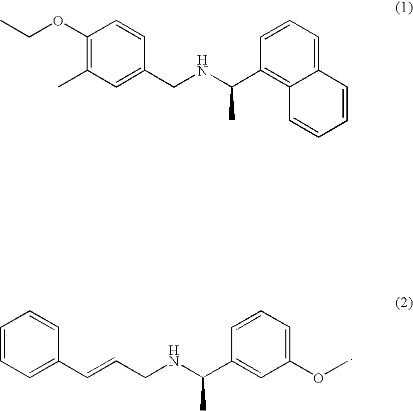

It is another aspect of the present invention to provide the prophylactic or therapeutic agent as described above, wherein the peptide is selected from the group consisting of γ-Glu-X-Gly (X is an amino acid or an amino acid derivative), γ-Glu-Val-Y (Y is an amino acid or an amino acid derivative), γ-Glu-Ala, γ-Glu-Gly, γ-Glu-Cys, γ-Glu-Met, γ-Glu-Thr, γ-Glu-Val, γ-Glu-Orn, Asp-Gly, Cys-Gly, Cys-Met, Glu-Cys, Gly-Cys, Leu-Asp, γ-Glu-Met(O), γ-Glu-γ-Glu-Val, γ-Glu-Val-NH₂, γ-Glu-Val-ol, γ-Glu-Ser, γ-Glu-Tau, γ-Glu-Cys(S-Me)(O), γ-Glu-Leu, γ-Glu-Ile, γ-Glu-t-Leu, γ-Glu-Cys(S-Me), and combinations thereof.

It is another aspect of the present invention to provide the prophylactic or therapeutic agent as described above, in which X is selected from the group consisting of Cys(SNO), Cys(S-allyl), Gly, Cys(S-Me), Cys, Abu, t-Leu, Cle, Aib, Pen, and Ser; and Y is selected from the group consisting of Gly, Val, Glu, Lys, Phe, Ser, Pro, Arg, Asp, Met, Thr, His, Orn, Asn, Cys, and Gln.

It is another aspect of the present invention to provide the prophylactic or therapeutic agent as described above, wherein the peptide is selected from the group consisting of γ-Glu-Val-Gly and γ-Glu-t-Leu-Gly.

It is another aspect of the present invention to provide the prophylactic or therapeutic agent as described above, in which the peptide derivative has the structure γ-Glu-X—OCH(Z)CO₂H, and wherein X is an amino acid or an amino acid derivative, and Z is H or CH₃.

It is another aspect of the present invention to provide a compound having the structure γ-Glu-X—OCH(Z)CO₂H, wherein X is an amino acid or an amino acid derivative, and Z is H or CH₃;

It is another aspect of the present invention to provide the compound as described above, in which X is t-Leu or Abu.

It is another aspect of the present invention to provide the compound γ-Glu-t-Leu-Gly.

### BRIEF DESCRIPTION OF THE DRAWINGS

FIG. 1 shows a graph illustrating an action of calcium on a calcium receptor. The human calcium receptor cRNA was injected into Xenopus laevis oocytes by microinjection. Values of the intracellular response currents were recorded when a calcium chloride solution was added at an arbitrary concentration. The maximum value of the intracellular currents was defined as the response current value (maximum response value). It was confirmed that no response was observed in oocytes injected with distilled water by microinjection as a control.

FIG. 2 shows a graph illustrating an action of an L-amino acid on a calcium receptor. The human calcium receptor cRNA was injected into Xenopus laevis oocytes by microinjection. Values of intracellular response currents were recorded when a 10 mM L-amino acid solution was added. The maximum value of the intracellular currents was defined as the response current value. It was confirmed that no response was observed in oocytes injected with distilled water by microinjection as a control.

FIG. 3 shows a graph illustrating an action of a D-amino acid on a calcium receptor. The human calcium receptor cRNA was injected into Xenopus laevis oocytes by microinjection. Values of intracellular response currents were recorded when a 10 mM D-amino acid solution was added. The maximum value of the intracellular currents was defined as the response current value. It was confirmed that no response was observed in oocytes injected with distilled water by microinjection as a control.

FIG. 4 shows a graph illustrating an action of a peptide on a calcium receptor. The human calcium receptor cRNA was injected into Xenopus laevis oocytes by microinjection. Values of intracellular response currents were recorded when a peptide solution was added at an arbitrary concentration. The maximum value of the intracellular currents was defined as the response current value. It was confirmed that no response was observed in oocytes injected with distilled water by microinjection as a control.

FIG. 5 shows a graph illustrating a therapeutic effect on diarrhea of a peptide which is able to activate the calcium receptor. In a mouse 5-HTP-induced defecation model, samples having 0.1% and 1% of the peptide γEVG were able to improve stool formation in a dose-dependent manner.

FIG. 6 shows a graph illustrating the effect of γEVG on fluid absorption in a large intestine loop method.

FIG. 7 shows a graph illustrating the effects of GSH and γ-Glu-t-Leu-Gly on fluid absorption in a large intestine loop method.

FIG. 8 shows a graph illustrating the effect of cinacalcet in a large intestine loop method.

### DETAILED DESCRIPTION OF EXEMPLARY EMBODIMENTS

A prophylactic or therapeutic agent useful for treating diarrhea can contain a compound which is able to activate a calcium receptor.

The term “calcium receptor” can mean a receptor that is called the Calcium Sensing Receptor (CaSR) and belongs to class C of the seven-transmembrane receptors. The term “calcium receptor activator” can mean a substance that binds to, and as a result, activates the calcium receptor. The phrase “to activate a calcium receptor” or “activates the calcium receptor” can mean that a ligand that binds to the calcium receptor and activates a guanine nucleotide binding protein, and thereby transmits a signal. In addition, the term “calcium receptor activity” can mean that the calcium receptor transmits a signal.

<1> Compound Having Calcium Receptor-Activating Action

Examples of the compound that is able to activate a calcium receptor include a peptide, a derivative thereof, or various low molecular weight compounds. Such compounds can also be obtained by screening, such as by reacting a calcium receptor with a test substance and detecting calcium receptor activity. Then, it can be confirmed that the thus-obtained peptide or low molecular weight compound has a prophylactic or therapeutic effect on diarrhea.

Hereinafter, method steps for screening for compounds able to activate a calcium receptor are specifically described, but are not limited to these steps:

1) measure a calcium receptor activity by adding a test substance to a calcium receptor activity measurement system;

2) compare the calcium receptor activity with and without the test substance with calcium receptor activity; and

3) select the test substance which is able to activate the calcium receptor when the test substance is added.

The calcium receptor activity is, for example, measured by using a measurement system using cells that express calcium receptors. These cells can be cells that endogenously express calcium receptors, or can be recombinant cells into which an exogenous calcium receptor gene is introduced. The measurement system for determining calcium receptor activity can be used without any particular limitation as long as, when an extracellular ligand (activator) specific to a calcium receptor is added to the cells that express calcium receptors, the measurement system can detect the binding (reaction) between the activator and the calcium receptor, or can respond to the binding (reaction) between the activator and the calcium receptor to thereby transmit a detectable signal into the cells. When calcium receptor activity is detected via the reaction with the test substance, the test substance is said to be able to activate or stimulate a calcium receptor, and can have a prophylactic or therapeutic effect on diarrhea.

The prophylactic or therapeutic effect on diarrhea can be confirmed by a test or the like, using an anticancer agent-induced diarrhea model as described in the examples, a mouse 5-HTP-induced defecation model, or the like. Furthermore, the compounds to be used as test substances are not particularly limited. However, the peptide can be of 2 to 10 amino acid residues, or a derivative thereof, and in another example, can be of 2 or 3 amino acid residues or a derivative thereof. The amino acid residue at the N-terminal side of the peptide can be γ-glutamic acid.

The origin of the calcium receptor is not particularly limited. Examples thereof include not only the human calcium receptor, but also calcium receptors derived from, or native to, an animal such as a mouse, a rat, and a dog. Specifically, examples of the calcium receptor can include the human calcium receptor encoded by the human calcium receptor gene registered with GenBank Accession No NM_{—}000388. The calcium receptor is not limited to the protein encoded by the gene having this sequence, and can be a protein encoded by a gene which is 60% or more, in another example 80% or more, and in another example 90% or more homology to the GenBank sequence, as long as the gene encodes a protein having the function of the calcium receptor. The GPRC6A receptor, also called the 5.24 receptor, is also known as a subtype of the calcium receptor, and can be used. It should be noted that the calcium receptor function can be confirmed by expressing the genes in cells and measuring the change in the current when calcium is added, and the change in the intracellular calcium ion concentration.

As described above, calcium receptor activity can be confirmed by using live cells expressing a calcium receptor or its fragment, cell membranes expressing a calcium receptor or its fragment, an in vitro system containing the calcium receptor or its fragment, or the like.

An example using live cells is described below. However, confirmation of the calcium receptor activity is not limited to this example.

The calcium receptor can be expressed in cultured cells such as Xenopus laevis oocytes, hamster ovarian cells, and human fetal kidney cells. The calcium receptor can be expressed by cloning the calcium receptor gene in a plasmid that carries a foreign gene, and introducing the plasmid or cRNA into the cells. To detect the reaction, an electrophysiological technique and a fluorescent indicator that indicates an increase in the intracellular calcium level can be used.

Expression of the calcium receptor can be first confirmed based on the response to calcium or a known activator. Oocytes in which intracellular current is observed in response to 5 mM of calcium, or cultured cells in which fluorescence of the fluorescent indicator reagent is observed in response to 5 mM of calcium, can be used. The calcium concentration dependency is determined by changing the calcium concentration. Then, a test substance such as a peptide is prepared to a concentration of about 1 μM to 1 mM, and added to the oocytes or cultured cells, and the calcium receptor activity of the peptide is determined.

Examples of the compound that is able to activate a calcium receptor include various peptides or derivatives thereof, or various low molecular weight compounds. Hereinafter, when the term “peptide” is used, it can sometimes means either a peptide or a peptide derivative. Examples of the peptide include γ-Glu-X-Gly where X represents an amino acid or an amino acid derivative, γ-Glu-Val-Y where Y represents an amino acid or an amino acid derivative, γ-Glu-Ala, γ-Glu-Gly, γ-Glu-Cys, γ-Glu-Met, γ-Glu-Thr, γ-Glu-Val, γ-Glu-Orn, Asp-Gly, Cys-Gly, Cys-Met, Glu-Cys, Gly-Cys, Leu-Asp, γ-Glu-Met(O), γ-Glu-γ-Glu-Val, γ-Glu-Val-NH₂, γ-Glu-Val-ol, γ-Glu-Ser, γ-Glu-Tau, γ-Glu-Cys(S-Me)(O), γ-Glu-Leu, γ-Glu-Ile, γ-Glu-t-Leu, and γ-Glu-Cys(S-Me).

Further, the peptide can be a peptide derivative having a structure of γ-Glu-X—OCH(Z)CO₂H where X represents an amino acid or an amino acid derivative, and Z represents H (a hydrogen atom) or CH₃ (a methyl group). Specific examples include γ-Glu-Val-GlyA, γ-Glu-t-Leu-GlyA, γ-Glu-Abu-GlyA, γ-Glu-Val-LacA, γ-Glu-t-Leu-LacA, and γ-Glu-Abu-LacA. It should be noted that GlyA represents glycolic acid and LacA represents lactic acid. Lactic acid may be S-lactic acid and/or R-lactic acid. Structural formulae of these compounds are described below.

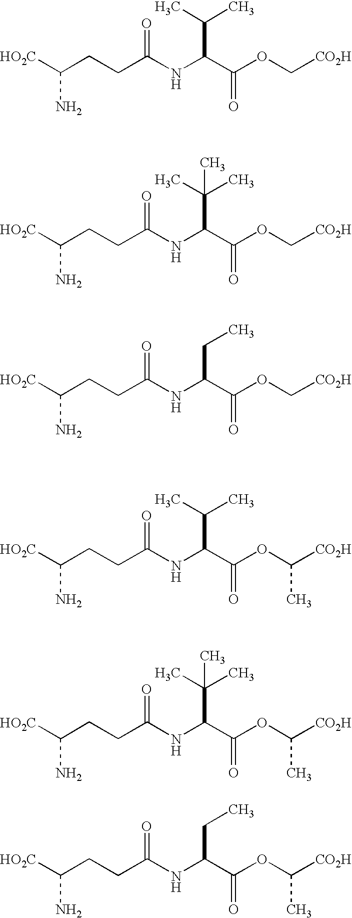

In the above formulas, preferably, X can represent Cys(SNO), Cys(S-allyl), Gly, Cys(S-Me), Cys, Abu, t-Leu, Cle, Aib, Pen, or Ser; and Y can represent Gly, Val, Glu, Lys, Phe, Ser, Pro, Arg, Asp, Met, Thr, His, Orn, Asn, Cys, Gln, GlyA, or LacA. Further preferably, examples of the compounds can be γ-Glu-Val-Gly and γ-Glu-t-Leu-Gly.

Amino acids can be L-amino acids, unless otherwise stated. Examples of the amino acid include a neutral amino acid such as Gly, Ala, Val, Leu, Ile, Ser, Thr, Cys, Met, Asn, Gln, Pro, and Hyp, an acidic amino acid such as Asp and Glu; a basic amino acid such as Lys, Arg, and His; an aromatic amino acid such as Phe, Tyr, and Trp; and homoserine, citrulline, ornithine, α-aminobutyric acid, norvaline, norleucine, and taurine. The amino acid may also be a non-naturally occurring (non-protein constituent) amino acid such as tert-leucine, cycloleucine, α-aminoisobutyric acid, and L-penicillamine. It should be noted that X in the peptide γ-Glu-X-Gly can be any one of the above-described amino acids or a derivative thereof, and can be an amino acid or a derivative thereof, other than Cys.

Herein, abbreviations for amino residues are as follows:

(1) Gly: Glycine

(2) Ala: Alanine

(3) Val: Valine

(4) Leu: Leucine

(5) Ile: Isoleucine

(6) Met: Methionine

(7) Phe: Phenylalanine

(8) Tyr: Tyrosine

(9) Trp: Tryptophan

(10) His: Histidine

(11) Lys: Lysine

(12) Arg: Arginine

(13) Ser: Serine

(14) Thr: Threonine

(15) Asp: Aspartic acid

(16) Glu: Glutamic acid

(17) Asn: Asparagine

(18) Gln: Glutamine

(19) Cys: Cysteine

(20) Pro: Proline

(21) Orn: Ornithine

(22) Sar: Sarcosine

(23) Cit: Citrulline

(24) N-Val: Norvaline

(25) N-Leu: Norleucine

(26) Abu: α-Aminobutyric acid

(27) Tau: Taurine

(28) Hyp: Hydroxyproline

(29) t-Leu: tert-Leucine

(30) Cle: Cycloleucine

(31) Aib: α-Aminoisobutyric acid (2-methylalanine)

(32) Pen: L-Penicillamine

Examples of amino acid derivatives include various derivatives of the above amino acids such as an unusual amino acid, a non-natural amino acid, an amino alcohol, and a substituted amino acid with a side chain such as the terminal carbonyl group, the terminal amino group, and the thiol group of cysteine, that can contain various substituents. Examples of the substituents include an alkyl group, an acyl group, a hydroxy group, an amino group, an alkylamino group, a nitro group, a sulfonyl group, and various protection groups. Examples of the substituted amino acid include Arg(NO₂): N-γ-nitroarginine; Cys(SNO): S-nitrocysteine; Cys(S-Me): S-methylcysteine; Cys(S-allyl): S-allylcysteine; Val-NH₂: valinamide; and Val-ol: valinol (2-amino-3-methyl-1-butanol).

It should be noted that γ-Glu-Cys(SNO)-Gly has the following structural formula, and the “(O)” in the above formulae γ-Glu-Met(O) and γ-Glu-Cys(S-Me)(O) can indicate a sulfoxide structure.

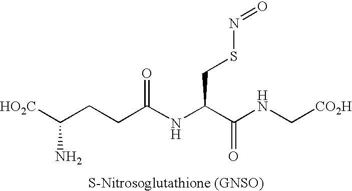

γ-Glu-X-Gly where X can be an amino acid or an amino acid derivative, γ-Glu-Val-Y where Y can be an amino acid or an amino acid derivative, γ-Glu-Ala, γ-Glu-Gly, γ-Glu-Cys, γ-Glu-Met, γ-Glu-Thr, γ-Glu-Val, γ-Glu-Orn, Asp-Gly, Cys-Gly, Cys-Met, Glu-Cys, Gly-Cys, Leu-Asp, γ-Glu-Met(O), γ-Glu-γ-Glu-Val, γ-Glu-Val-NH₂, γ-Glu-Val-ol, γ-Glu-Ser, γ-Glu-Tau, γ-Glu-Cys(S-Me)(O), γ-Glu-Leu, γ-Glu-Ile, γ-Glu-t-Leu, and γ-Glu-Cys(S-Me) can each activate the calcium receptor. Therefore, γ-Glu-X-Gly where X can be an amino acid or an amino acid derivative, γ-Glu-Val-Y where Y can be an amino acid or an amino acid derivative, γ-Glu-Ala, γ-Glu-Gly, γ-Glu-Cys, γ-Glu-Met, γ-Glu-Thr, γ-Glu-Val, γ-Glu-Orn, Asp-Gly, Cys-Gly, Cys-Met, Glu-Cys, Gly-Cys, Leu-Asp, γ-Glu-Met(O), γ-Glu-γ-Glu-Val, γ-Glu-Val-NH₂, γ-Glu-Val-ol, γ-Glu-Ser, γ-Glu-Tau, γ-Glu-Cys(S-Me)(O), γ-Glu-Leu, γ-Glu-Ile, γ-Glu-t-Leu, and γ-Glu-Cys(S-Me) can each be used as a therapeutic agent for diarrhea. The chosen peptide can be used alone or can be used in a random mixture of two or more peptides.

A commercially available peptide product can be used. Furthermore, the peptide can be obtained by appropriately using a known technique such as chemical synthesis, or by synthesizing the peptide by an enzymatic reaction. Since the number of amino acid residues which make up the peptide is usually small, such as 2 or 3 residues, chemically synthesizing the peptide can be convenient. When chemically synthesizing the peptide, the oligopeptide can be synthesized or semi-synthesized by using a peptide synthesizer. Chemically synthesizing the peptide includes synthesizing the peptide by a solid phase synthetic method. The peptide synthesized as described above can be purified by usual means such as ion exchange chromatography, reversed phase high performance liquid chromatography, or affinity chromatography. Solid phase synthesis of the peptide and the subsequent peptide purification are well known in the technical field.

The peptide can also be produced by an enzymatic reaction. For example, the method described in WO 2004/011653 can be used. That is, the peptide can also be produced by reacting one amino acid or dipeptide having an esterified or amidated carboxyl terminus with an amino acid having a free amino group (for example, an amino acid with a protected carboxygroup) in the presence of a peptide-producing enzyme, and purifying the produced dipeptide or tripeptide. The peptide-producing enzyme can be a part of a culture of a microorganism having the ability to produce the peptide, microbial cells separated from the culture, or a processed product of cells of the microorganism, or a peptide-producing enzyme derived from the microorganism.

Examples of the low molecular weight compound include cinacalcet ((R)—N-(3-(3-(trifluoromethyl)phenyl)propyl)-1-(1-naphthyl)ethylamine) and analogous compounds thereof. Examples of an analogous compound of cinacalcet include the compound represented by the chemical formula (1) ((R)—N-[(4-ethoxy-3-methylphenyl)methyl]-1-(1-naphthyl)ethylamine)), the compound represented by the chemical formula (2) ((R)—N-(3-phenylprop-2-enyl)-1-(3-methoxyphenyl)ethylamine), or the like. These compounds may be synthesized by a known method, such as described in U.S. Pat. No. 6,211,244, for example. Furthermore, commercially available products may also be used.

The compound can also be in the form of a salt. When the peptide is in the form of a salt, the salt may be a pharmacologically acceptable salt. Examples of a salt with an acidic group such as a carboxyl group in the formula include an ammonium salt, a salt with an alkali metal such as sodium and potassium, a salt with an alkaline earth metal such as calcium and magnesium, an aluminum salt, a zinc salt, a salt with an organic amine such as triethylamine, ethanolamine, morpholine, pyrrolidine, piperidine, piperazine, and dicyclohexylamine, and a salt with a basic amino acid such as arginine and lysine. Examples of a salt with a basic group include a salt with an inorganic acid such as hydrochloric acid, sulfuric acid, phosphoric acid, nitric acid, and hydrobromic acid; a salt with an organic carboxylic acid such as acetic acid, citric acid, benzoic acid, maleic acid, fumaric acid, tartaric acid, succinic acid, tannic acid, butyric acid, hibenzoic acid, pamoic acid, enanthoic acid, decanoic acid, teoclic acid, salicylic acid, lactic acid, oxalic acid, mandelic acid, and malic acid; and a salt with an organic sulfonic acid such as methanesulfonic acid, benzenesulfonic acid, and p-toluenesulfonic acid.

<2> Prophylactic or Therapeutic Agent for Diarrhea

The compound that is able to activate a calcium receptor can be used as an active ingredient in a prophylactic or therapeutic agent for diarrhea. Examples of the form of the prophylactic or therapeutic agent for diarrhea include pharmaceuticals, quasi-drugs, and foods.

The method of administering the prophylactic or therapeutic agent for diarrhea is not particularly limited, and can include oral administration, an invasive administration utilizing an injection, a suppository administration, or a transdermal administration. The prophylactic or therapeutic agent for diarrhea can be administered in the form of a conventional pharmaceutical formulation by mixing the active ingredient with a nontoxic solid or liquid pharmaceutical carrier, which is suitable for oral or injectable administration. Examples of these formulations include a solid formulation such as a tablet, a granule, a powder, and a capsule; a liquid formulation such as a solution, a suspension, and an emulsion; and a lyophilizate or the like. These formulations may be prepared by known methods.

Examples of nontoxic carriers for pharmaceuticals include glucose, lactose, sucrose, starch, mannitol, dextrin, fatty acid glyceride, polyethylene glycol, hydroxyethyl starch, ethylene glycol, polyoxyethylene sorbitan fatty acid ester, gelatin, albumin, amino acid, water, and physiological saline. Furthermore, if required, a conventional agent such as a stabilizing agent, a wetting agent, an emulsifier, a binder, and a tonicity agent may be appropriately added.

The compound which is able to activate a calcium receptor to be used for the prophylactic or therapeutic agent for diarrhea is a peptide or a low molecular weight compound as described herein, and can be a known compound which is able to activate a calcium receptor. Furthermore, the prophylactic or therapeutic agent for diarrhea can contain, in addition to the peptide and/or the low molecular weight compound, one or more known calcium receptor activators.

Examples of known calcium receptor activators include, but are not limited to, a cation such as a calcium cation and a gadolinium cation; a basic peptide such as polyarginine and polylysine; a polyamine such as putrescine, spermine, and spermidine; a protein such as protamine; an amino acid such as phenylalanine; a peptide such as glutathione; and an analogous compound as cinacalcet. The known calcium receptor activator can be added alone or may be added as a mixture of any two or more.

When a known calcium receptor activator is mixed with the peptide or the low molecular weight compound as described herein, stronger activation of the calcium receptor can be observed. When a peptide is used as the calcium receptor activator, the ratio of the peptide to the known calcium receptor activator is not particularly limited as long as stronger activation of the calcium receptor is achieved. For example, the mass ratio of the known calcium receptor activator to the peptide can be 1:100 to 100:1.

The amount of the prophylactic or therapeutic agent for diarrhea to be administered can be any amount as long as the amount is effective for therapy or prophylaxis, and is appropriately adjusted depending on the age, sex, body weight, symptom, and the like of the patient. For example, when administering orally, the total amount of the peptide can be 0.01 g to 10 g per kg body weight per dose, and in another example 0.1 g to 1 g per kg body weight per dose. The frequency of administration is not particularly limited, and can be once to several times per day.

The amount of the compound that is able to activate a calcium receptor in the prophylactic or therapeutic agent for diarrhea is not limited as long as the amount is consistent with the above-described dosage. The amount can be 0.000001% by mass to 99.9999% by mass, and in another example 0.00001% by mass to 99.999% by mass, and in another example 0.0001% by mass to 99.99% by mass, with respect to the dry weight.

The prophylactic or therapeutic agent for diarrhea can also be in the form of a food or drink. For example, the prophylactic or therapeutic agent can be formulated into a food or drink in a container or packaging which indicates that the agent has a therapeutic or prophylaxis effect for diarrhea. The form of the food or drink is not particularly limited, and the food or drink may be produced using the production method that is usually used, and with the same materials, except that the compound which is able to activate a calcium receptor is blended. Examples of the food include a seasoning, a drink such as juice or cow milk, a confectionery, a jelly, a health food, a processed agricultural product, a processed fishery product, a processed animal product such as cow milk, and a food supplement. Further, examples of diarrhea include irritable bowel syndrome, functional diarrhea, inflammatory bowel disease, tympanitis, bacterial diarrhea, and dyspepsia.

### EXAMPLES

Hereinafter, the present invention is more specifically described with reference to the following non-limiting examples.

### Example 1

### Preparation of a Calcium Receptor Gene (cRNA)

The gene encoding the calcium receptor was prepared as follows. On the basis of the DNA sequence registered at NCBI (calcium receptor: NM_{—}000388), synthetic oligo DNAs (forward primer (SEQ ID NO: 1) and reverse primer (SEQ ID NO: 2)) were synthesized.

Human kidney cDNA (manufactured by Clontech) was used as a source, and PCR was performed by using the primers and Pfu ultra DNA Polymerase (manufactured by Stratagene) under the following conditions. After a reaction at 94° C. for 3 minutes, a cycle of reactions at 94° C. for 30 seconds, 55° C. for 30 seconds, and 72° C. for 2 minutes was repeated 35 times, and then a reaction was performed at 72° C. for 7 minutes. Whether amplification was attained by PCR was detected by performing agarose electrophoresis, staining with a DNA staining reagent, and subsequent ultraviolet irradiation. The chain lengths of the PCR products were confirmed by comparison with DNA markers of known sizes which were simultaneously subjected to the electrophoresis. The plasmid vector pBR322 was digested with the restriction enzyme EcoRV (manufactured by Takara). The gene fragment amplified by PCR was ligated to the cleavage site of the plasmid by using Ligation Kit (manufactured by Promega). The Escherichia coli DH5α strain was transformed with each ligation reaction solution, and a transformant harboring the plasmid in which the PCR amplification product was cloned was selected. The PCR amplification product was confirmed by DNA sequence analysis. By using the recombinant plasmid as a template, cRNA of the calcium receptor gene was prepared using a cRNA preparation kit (manufactured by Ambion).

### Example 2

### Preparation of Various Samples

As L-amino acid samples, 23 kinds of special grade amino acids including alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, and taurine (all from Ajinomoto Co., Inc.), and hydroxyproline (Nacarai Tesque, Inc.), were used. For D-Cys and D-Trp (Nacarai Tesque, Inc.) and calcium chloride, a special grade was used.

Furthermore, as peptide specimens, γ-Glu-Cys-Gly (Sigma Aldrich Japan K.K.), γ-Glu-Cys(SNO)-Gly (Dojindo Laboratories), γ-Glu-Ala (Bachem Feinchemikalien AG), γ-Glu-Gly (Bachem Feinchemikalien AG), γ-Glu-Cys (Sigma Aldrich Japan K.K.), γ-Glu-Met (Bachem Feinchemikalien AG), γ-Glu-Abu-Gly (Abu: α-aminobutyric acid, Bachem Feinchemikalien AG), γ-Glu-Thr (Kokusan Chemical Co., Ltd.), γ-Glu-Val (Kokusan Chemical Co., Ltd.), γ-Glu-Leu (custom synthesis product), γ-Glu-Ile (custom synthesis product), γ-Glu-Orn (Kokusan Chemical Co., Ltd.), Asp-Gly (custom synthesis product), Cys-Gly (custom synthesis product), Cys-Met (custom synthesis product), Glu-Cys (custom synthesis product), Gly-Cys (custom synthesis product), Leu-Asp (custom synthesis product), γ-Glu-Val-Val (custom synthesis product), γ-Glu-Val-Glu (custom synthesis product), γ-Glu-Val-Lys (custom synthesis product), γ-Glu-γ-Glu-Val (custom synthesis product), γ-Glu-Gly-Gly (custom synthesis product), γ-Glu-Val-Phe (custom synthesis product), γ-Glu-Val-Ser (custom synthesis product), γ-Glu-Val-Pro (custom synthesis product), γ-Glu-Val-Arg (custom synthesis product), γ-Glu-Val-Asp (custom synthesis product), γ-Glu-Val-Met (custom synthesis product), γ-Glu-Val-Thr (custom synthesis product), γ-Glu-Val-His (custom synthesis product), γ-Glu-Val-Asn (custom synthesis product), γ-Glu-Val-Gln (custom synthesis product), γ-Glu-Val-Cys (custom synthesis product), γ-Glu-Val-Orn (custom synthesis product), γ-Glu-Ser-Gly (custom synthesis product), and γ-Glu-Pen-Gly (custom synthesis product) were used. Glutamine and cysteine were prepared upon use, and the other samples were stored at −20° C. after preparation. Peptides having a purity of 90% or higher were used, except for γ-Glu-Cys, which was 80% or higher.

After dissolving each sample in solution, if the pH of the solution is either acidic or alkaline, the pH of the solution was adjusted to an approximately neutral pH by using NaOH or HCl. The solution used for dissolution of amino acids and peptides, preparation of Xenopus laevis oocytes, and culture of the oocytes had the following composition: 96 mM NaCl, 2 mM KCl, 1 mM MgCl₂, 1.8 mM CaCl₂, 5 mM Hepes, pH 7.2.

### Example 3

### Synthesis of γ-Glu-Val-Gly

Boc-Val-OH (8.69 g, 40.0 mmol) and Gly-OBzl.HCl (8.07 g, 40.0 mmol) were dissolved in methylene chloride (100 ml) and the solution was kept at 0° C. Triethylamine (6.13 ml, 44.0 mmol), HOBt (1-hydroxybenzotriazole, 6.74 g, 44.0 mmol), and WSC.HCl (1-ethyl-3-(3-dimethylaminopropyl)carbodiimide hydrochloride, 8.44 g, 44.0 mmol) were added to the solution, and the mixture was stirred overnight at room temperature. The reaction solution was concentrated under reduced pressure, and the residue was dissolved in ethyl acetate (200 ml). The solution was washed with water (50 ml), a 5% citric acid aqueous solution (50 ml×twice), saturated brine (50 ml), a 5% sodium bicarbonate aqueous solution (50 ml×twice), and saturated brine again (50 ml). The organic layer was dried over anhydrous magnesium sulfate, magnesium sulfate was removed by filtration, and the filtrate was concentrated under reduced pressure. The residue was recrystallized from ethyl acetate/n-hexane to obtain Boc-Val-Gly-OBzl (13.2 g, 36.2 mmol) as a white crystal.

Boc-Val-Gly-OBzl (5.47 g, 15.0 mmol) was added to a 4 NHCl/dioxane solution (40 ml), and the mixture was stirred at room temperature for 50 minutes. Dioxane was removed by concentration under reduced pressure, n-hexane (30 ml) was added to the residue, and the mixture was concentrated under reduced pressure. The procedure was repeated 3 times to quantitatively obtain H-Val-Gly-OBzl.HCl.

H-Val-Gly-OBzl.HCl and Z-Glu-OBzl (5.57 g, 15.0 mmol) described above were dissolved in methylene chloride (50 ml), and the solution was maintained at 0° C. Triethylamine (2.30 ml, 16.5 mmol), HOBt (1-hydroxybenzotriazole, 2.53 g, 16.5 mmol), and WSC.HCl (1-ethyl-3-(3-dimethylaminopropyl)carbodiimide hydrochloride, 3.16 g, 16.5 mmol) were added to the solution, and the mixture was stirred at room temperature overnight for 2 days. The reaction solution was concentrated under reduced pressure, and the residue was dissolved in heated ethyl acetate (1,500 ml). The solution was washed with water (200 ml), 5% citric acid aqueous solution (200 ml×twice), saturated brine (150 ml), 5% sodium bicarbonate aqueous solution (200 ml×twice), and saturated brine again (150 ml). The organic layer was dried over anhydrous magnesium sulfate, magnesium sulfate was removed by filtration, and the filtrate was concentrated under reduced pressure. The precipitated crystal was collected by filtration and dried under reduced pressure to obtain Z-Glu(Val-Gly-OBzl)-OBzl (6.51 g, 10.5 mmol) as a white crystal.

Z-Glu(Val-Gly-OBzl)-OBzl described above (6.20 g, 10.03 mmol) was suspended in ethanol (200 ml), 10% palladium/carbon (1.50 g) was added to the suspension, and a reduction reaction was performed under a hydrogen atmosphere at 55° C. for 5 hours. During the reaction, 100 ml in a total volume of water was gradually added. The catalyst was removed by filtration using a Kiriyama funnel (Kiriyama glass Co.), and the filtrate was concentrated under reduced pressure to a half volume. The reaction solution was further filtered through a membrane filter, and the filtrate was concentrated under reduced pressure. After the residue was dissolved in a small volume of water, ethanol was added to precipitate a crystal, and the crystal was collected by filtration and dried under reduced pressure to obtain γ-Glu-Val-Gly as a white powder (2.85 g, 9.40 mmol).

ESI-MS: (M+H)⁺=304.1.

¹H-NMR (400 MHz, D₂O) δ (ppm): 0.87 (3H, d, J=6.8 Hz), 0.88 (3H, d, J=6.8 Hz), 1.99-2.09 (3H, m), 2.38-2.51 (2H, m) 3.72 (1H, t, J=6.35 Hz), 3.86 (1H, d, J=17.8 Hz), 3.80 (1H, d, J=17.8 Hz), 4.07 (1H, d, J=6.8 Hz).

### Example 4

### Synthesis of γ-Glu-Cys(S-Me)-Gly [Cys(S-Me): S-methylcysteine

Reduced glutathione (15.0 g, 48.8 mmol) was added to water (45 ml) and sodium hydroxide (4.52 g, 2.2 equivalents, 107 mmol) was added portionwise to the mixture under bubbling with nitrogen. Methyl iodide (4.56 ml, 1.5 equivalents, 73 mmol) was added to the mixture, and the solution was sealed and stirred at room temperature for 2 hours. The reaction solution was adjusted to pH 2 to 3 with concentrated hydrochloric acid, supplemented with ethanol (150 ml), and stored overnight in a refrigerator. Since an oily product separated, the supernatant was removed. When the remaining oily product was dissolved in water and gradually supplemented with ethanol, a partially crystallized oily product precipitated. Therefore, the supernatant was removed again. The residue was dissolved in water (300 ml), adsorbed to a column filled with an ion exchange resin (Dowex 1-acetate, 400 ml), washed with water, and then eluted with a 1 N acetic acid aqueous solution. The eluate was concentrated under reduced pressure, and reprecipitated from water/ethanol to obtain γ-Glu-Cys(S-Me)-Gly as a white powder (5.08 g, 15.8 mmol).

FAB-MS: (M+H)⁺=322.

¹H-NMR (400 MHz, D₂O) δ (ppm): 2.14 (3H, s), 2.15-2.22 (2H, m), 2.50-2.58 (2H, m), 2.86 (1H, dd, J=9.0 Hz, J=14.0 Hz), 3.03 (1H, dd, J=5.0 Hz, J=14.0 Hz), 3.84 (1H, t, J=6.5 Hz), 3.99 (2H, s), 4.59 (1H, dd, J=5.0 Hz, J=9.0 Hz)

### Example 5

### Synthesis of Other Peptides

γ-Glu-Met(O), γ-Glu-Val-NH₂, γ-Glu-Val-ol, γ-Glu-Ser, γ-Glu-Tau, γ-Glu-Cys(S-Me)(O), γ-Glu-t-Leu, γ-Glu-Cys(S-allyl)-Gly, γ-Glu-Cys(S-Me), γ-Glu-Cle-Gly, and γ-Glu-Aib-Gly were synthesized in accordance with Examples 3 and 4.

### Example 6

### Synthesis of γ-Glu-t-Leu-Gly

Boc-t-Leu-OH (9.26 g, 40.0 mmol) and Gly-OBzl-HCl (8.06 g, 40.0 mmol) were dissolved in methylene chloride (60 ml) and the solution was kept at 0° C. Triethylamine (5.60 ml, 40.0 mmol), HOBt (1-hydroxybenzotriazole, 6.75 g, 44.0 mmol), and WSC.HCl (1-ethyl-3-(3-dimethylaminopropyl)carbodiimide hydrochloride, 8.47 g, 44.0 mmol) were added to the solution, and the mixture was stirred overnight at room temperature. The reaction solution was concentrated under reduced pressure, and the residue was dissolved in ethyl acetate (200 ml). The solution was washed with water (50 ml), 5% citric acid aqueous solution (50 ml×twice), saturated brine (50 ml), 5% sodium bicarbonate aqueous solution (50 ml×twice), and saturated brine again (50 ml). The organic layer was dried over anhydrous magnesium sulfate, magnesium sulfate was removed by filtration, and the filtrate was concentrated under reduced pressure. The residue was recrystallized from ethyl acetate/n-hexane to obtain Boc-t-Leu-Gly-OBzl (15.20 g, 40.1 mmol) as a viscous, oily product.

Boc-t-Leu-Gly-OBzl (15.20 g, 40.1 mmol) was added to a 4 N HCl/dioxane solution (200 ml), and the mixture was stirred at room temperature for 1 hour. Dioxane was removed by concentration under reduced pressure, n-hexane (30 ml) was added to the residue, and the mixture was concentrated under reduced pressure. The procedure was repeated 3 times to quantitatively obtain H-t-Leu-Gly-OBzl.HCl

H-t-Leu-Gly-OBzl.HCl and Z-Glu-OBzl (14.93 g, 40.2 mmol) described above were dissolved in methylene chloride (80 ml), and the solution was kept at 0° C. Triethylamine (5.60 ml, 40.2 mmol), HOBt (6.79 g, 44.2 mmol), and WSC.HCl (8.48 g, 44.2 mmol) were added to the solution, and the mixture was stirred at room temperature overnight for 2 days. The reaction solution was concentrated under reduced pressure, and the residue was dissolved in heated ethyl acetate (300 ml). The solution was washed with water (100 ml), 5% citric acid aqueous solution (100 ml×twice), saturated brine (100 ml), 5% sodium bicarbonate aqueous solution (100 ml×twice), and saturated brine again (100 ml). The organic layer was dried over anhydrous magnesium sulfate, magnesium sulfate was removed by filtration, and the filtrate was concentrated under reduced pressure. The residue was purified by silica gel chromatography to obtain Z-Glu(t-Leu-Gly-OBzl)-OBzl (16.10 g, 25.5 mmol) as a viscous, oily product.

Z-Glu(t-Leu-Gly-OBzl)-OBzl described above (16.10 g, 25.5 mmol) was suspended in ethanol (300 ml), 10% palladium carbon (2.00 g) was added to the suspension, and a reduction reaction was performed under a hydrogen atmosphere at room temperature for 5 hours. During the reaction, 100 ml in a total volume of water were gradually added. The catalyst was removed by filtration using a Kiriyama funnel (Kiriyama glass Co.), and the filtrate was concentrated under reduced pressure to a half volume. The reaction solution was further filtered through a membrane filter, and the filtrate was concentrated under reduced pressure. After the residue was dissolved in a small volume of water, ethanol was added to the precipitate a crystal, and the crystal was collected by filtration and freeze dried to obtain γ-Glu-t-Leu-Gly as a white powder (6.70 g, 21.1 mmol).

ESI-MS: (M+H)⁺318.10.

¹H-NMR (400 MHz, D₂O) δ (ppm): 0.95 (9H, s), 2.04-2.08 (2H, m), 2.45-2.48 (2H, m), 3.73 (1H, t), 3.87-3.90 (2H, m), 4.07 (1H, s).

### Example 7

### Synthesis of γ-Glu-Abu-GlyA

Boc-Abu-OH (6.10 g, 30.0 mmol) and benzyl glycolate (H-GlyA-OBzl, 4.39 g, 30.0 mmol) were dissolved in methylene chloride (40 ml) and the solution was kept at 0° C. DMAP (4-dimethylaminopyridine, 1.10 g, 9.0 mmol) and WSC.HCl (6.33 g, 33.0 mmol) were added to the solution, and the mixture was stirred at room temperature overnight. The reaction solution was concentrated under reduced pressure, and the residue was dissolved in ethyl acetate (150 ml). The solution was washed with water (50 ml), 5% citric acid aqueous solution (50 ml×twice), saturated brine (50 ml), 5% sodium bicarbonate aqueous solution (50 ml×twice), and saturated brine again (50 ml). The organic layer was dried over anhydrous magnesium sulfate, magnesium sulfate was removed by filtration, and the filtrate was concentrated under reduced pressure to obtain Boc-Abu-GlyA-OBzl (9.47 g, 28.1 mmol) as a viscous, oily product.

To the above-mentioned residue, a 4 N HCl/dioxane solution (141 ml) was added, and the mixture was stirred at room temperature for 1 hour. Dioxane was removed by concentration under reduced pressure, n-Hexane (30 ml) was added to the residue, and the mixture was concentrated under reduced pressure. The procedure was repeated 3 times to quantitatively obtain H-Abu-GlyA-OBzl.HCl.

H-Abu-GlyA-OBzl.HCl and Z-Glu-OBzl (10.47 g, 28.1 mmol) described above were dissolved in methylene chloride (100 ml) and the solution was maintained at 0° C. Triethylamine (4.30 ml, 30.9 mmol), HOBt (4.74 g, 30.9 mmol), and WSC.HCl (5.95 g, 30.9 mmol) were added to the solution, and the mixture was stirred at room temperature overnight for 2 days. The reaction solution was concentrated under reduced pressure, and the residue was dissolved in ethyl acetate (200 ml). The solution was washed with water (50 ml), 5% citric acid aqueous solution (150 ml×twice), saturated brine (50 ml), 5% sodium bicarbonate aqueous solution (50 ml×twice), and saturated brine again (50 ml). The organic layer was dried over anhydrous magnesium sulfate, magnesium sulfate was removed by filtration, and the filtrate was concentrated under reduced pressure. The residue was purified by silica gel chromatography to obtain Z-Glu(Abu-GlyA-OBzl)-OBzl (11.20 g, 19.0 mmol) as a viscous, oily product.

Z-Glu(Abu-GlyA-OBzl)-OBzl (11.20 g, 19.0 mmol) described above was suspended in ethanol (150 ml), 10% palladium carbon (2.00 g) was added to the suspension, and a reduction reaction was performed under a hydrogen atmosphere at room temperature for 5 hours. During the reaction, 50 ml in a total volume of water were gradually added. The catalyst was removed by filtration using a Kiriyama funnel (Kiriyama glass Co.) and the filtrate was concentrated under reduced pressure to a half volume. The reaction solution was further filtered through a membrane filter, and the filtrate was concentrated under reduced pressure. The residue was dissolved in water and freeze-dried to obtain γ-Glu-Abu-GlyA (5.00 g, 17.2 mmol) as a white powder.

ESI-MS: (M+H)⁺=291.10.

¹H-NMR (400 MHz, D₂O) δ (ppm): 0.86 (3H, t, J=7.40 Hz), 1.60-1.74 (1H, m), 1.82-1.88 (1H, m), 2.04-2.12 (2H, m), 2.45 (2H, t, J=7.40 Hz), 3.79 (1H, t, J=6.36 Hz), 4.31-4.45 (1H, m), 4.57 (2H, s).

### Example 8

### Synthesis of γ-Glu-Val-GlyA

γ-Glu-Val-GlyA was obtained as a white powder in 77.5% yield in the same manner as that in Example 7 except that Boc-Val-OH was used in place of Boc-Abu-OH.

ESI-MS: (M−H)⁻=303.20.

¹H-NMR (400 MHz, D₂O) δ (ppm): 0.90 (6H, t, J=6.52 Hz), 2.05-2.15 (2H, m), 2.15-2.25 (1H, m), 2.45-2.50 (2H, m), 3.80 (1H, t, J=6.52 Hz), 4.36 (1H, t, J=5.64 Hz), 4.61 (2H, s).

### Example 9

### Synthesis of γ-Glu-t-Leu-GlyA

γ-Glu-t-Leu-GlyA was obtained as a white powder in 73.4% yield in the same manner as that in Example 7 except that Boc-t-Leu-OH was used in place of Boc-Abu-OH.

ESI-MS: (M+H)⁺=319.20.

¹H-NMR (400 MHz, D₂O) δ (ppm): 0.94 (9H, s), 2.03-2.10 (2H, m), 2.45-2.50 (2H, m), 3.78 (1H, t), 4.26 (1H, s), 4.60 (2H, s).

### Example 10

### Synthesis of γ-Glu-Abu-LacA

γ-Glu-Abu-LacA was obtained as a white powder in 99.0% yield in the same manner as that in Example 7 except that benzyl (S)-lactate (H-LacA-OBzl) was used in place of benzyl glycolate (H-GlyA-OBzl).

ESI-MS: (M+H)⁺=305.10.

¹H-NMR (400 MHz, D₂O) δ (ppm): 0.91 (3H, t, J=7.40 Hz), 1.40 (3H, d, J=7.08 Hz), 1.60-1.75 (1H, m), 1.80-1.90 (1H, m), 2.00-2.12 (2H, m), 2.40-2.45 (2H, m), 3.74-3.78 (1H, m), 4.25-4.29 (1H, m), 4.89-4.95 (1H, m).

### Example 11

### Synthesis of γ-Glu-Val-LacA

γ-Glu-Val-LacA was obtained as a white powder in 78.0% yield in the same manner as that in Example 7 except that Boc-Val-OH was used in place of Boc-Abu-OH and benzyl (S)-lactate (H-LacA-OBzl) was used in place of benzyl glycolate (H-GlyA-OBzl).

ESI-MS: (M+H)⁺=319.20.

¹H-NMR (400 MHz, D₂O) δ (ppm): 0.85-0.92 (6H, m), 1.42 (3H, d, J=7.08 Hz), 2.02-2.11 (3H, m), 2.18-2.25 (1H, m), 2.42-2.50 (2H, m), 3.78 (1H, t, J=6.36 Hz), 4.20-4.31 (1H, m), 4.91-4.97 (1H, m).

### Example 12

### Synthesis of γ-Glu-t-Leu-LacA

γ-Glu-t-Leu-LacA was obtained as a white powder in 55.0% yield in the same manner as that in Example 7 except that Boc-t-Leu-OH was used in place of Boc-Abu-OH and benzyl (S)-lactate (H-LacA-OBzl) was used in place of benzyl glycolate (H-GlyA-OBzl).

ESI-MS: (M+H)⁺=333.20.

¹H-NMR (400 MHz, D₂O) δ (ppm): 0.96 (9H, s), 1.42 (3H, d, J=7.08 Hz), 2.05-2.10 (2H, m), 2.40-2.50 (2H, m), 3.73-3.78 (1H, m), 4.19 (1H, s), 4.90-5.00 (1H, m).

### Example 13

### Evaluation of Calcium Receptor-Activating Action

For evaluation of the calcium receptor-activating action, a Ca ion concentration-dependent Cl ionic current measuring method using a Xenopus laevis oocyte expression system was used. If each activator is added to Xenopus laevis oocytes expressing the calcium receptor, intracellular Ca ions increase. Then, the Ca ion concentration-dependent Cl channel opens, and the intracellular current value changes as an ionic current. By measuring the change in the intracellular current value, whether the calcium receptor-activating action is present or not can be determined.

Specifically, the abdomen of Xenopus laevis was opened, and an egg batch was taken out and then treated with a 1% collagenase solution at 20° C. for 2 hours to obtain individual oocytes. Into the oocytes, 50 nl of 1 μg/μl receptor cRNA or 50 nl of sterilized water per oocyte were injected by using a micro glass capillary, and the oocytes were cultured at 18° C. for 2 to 3 days. For the culture, a solution obtained by adding 2 mM pyruvic acid, 10 U/ml penicillin, and 10 μg/ml streptomycin to the solution in Example 2 was used. After the culture, a test solution was added to the oocytes injected with cRNA or sterilized water. Electrophysiological measurement was performed by using an amplifier Geneclamp 500 (manufactured by Axon) and recording software AxoScope 9.0 (manufactured by Axon). The oocytes were membrane potential-clamped at −70 mV by the double electrode potential clamp method, and the intracellular current was measured via the Ca ion concentration-dependent Cl ion channel. The maximum value of the intracellular current was defined as the response current value.

### Example 14

### Evaluation of Calcium Receptor-Activating Action of Calcium

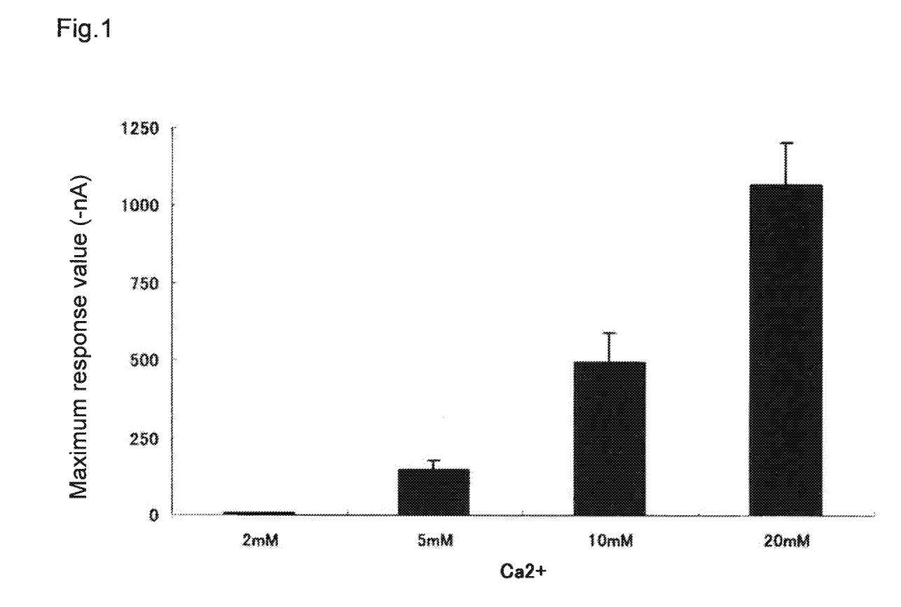

The calcium receptor-activating action of calcium was evaluated by using the method described in Example 13. That is, oocytes injected with cRNA of the calcium receptor or sterilized water were prepared, and membrane potential-clamped at −70 mV by the double electrode potential clamp method. To the potential-clamped oocytes, calcium was added (2 mM, 5 mM, 10 mM, and 20 mM), and Ca ion concentration-dependent Cl response current was measured. FIG. 1 shows the results. The results confirmed that cRNA of the calcium receptor injected into the oocytes was functionally expressed. Furthermore, since the oocytes injected with water did not respond to even a high concentration of calcium, it was confirmed that the calcium receptor was not expressed in the oocytes themselves.

### Example 15

### Evaluation of Calcium Receptor-Activating Action of L-amino Acids

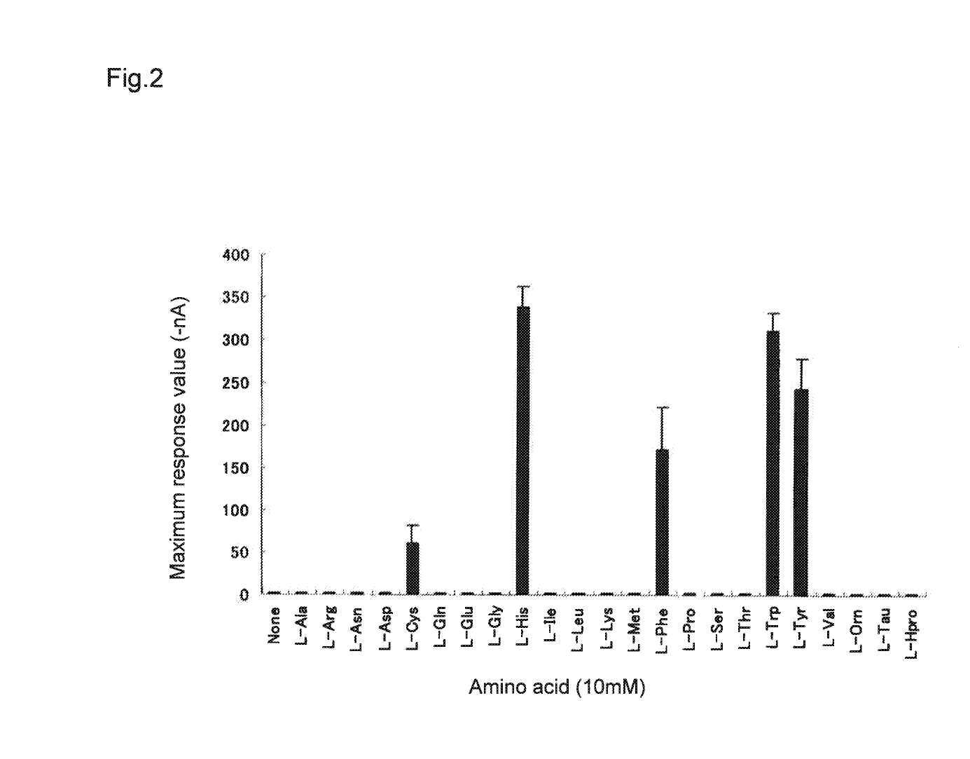

The calcium receptor-activating action of L-amino acids was evaluated by using the method described in Example 13. That is, oocytes injected with cRNA of the calcium receptor or sterilized water were prepared, and membrane potential-clamped at −70 mV by the double electrode potential clamp method. To the potential-clamped oocytes, alanine (10 mM), arginine (10 mM), asparagine (10 mM), aspartic acid (10 mM), cysteine (10 mM), glutamine (10 mM), glutamic acid (10 mM), glycine (10 mM), histidine (10 mM), isoleucine (10 mM), leucine (10 mM), lysine (10 mM), methionine (10 mM), phenylalanine (10 mM), proline (10 mM), serine (10 mM), threonine (10 mM), tryptophan (10 mM), tyrosine (10 mM), valine (10 mM), ornithine (10 mM), taurine (10 mM), or hydroxyproline (10 mM) was added, and Ca ion concentration-dependent Cl response current was measured. FIG. 2 shows the results. The results demonstrated that cysteine, histidine, phenylalanine, tryptophan, and tyrosine each had a definite calcium receptor-activating action. As for the above-described amino acids, the activating action is reported in Proc. Natl. Acad. Sci. USA, Apr. 25, 2000, 97(9): 4814-9.

### Example 16

### Evaluation of Calcium Receptor-Activating Action of D-Cysteine

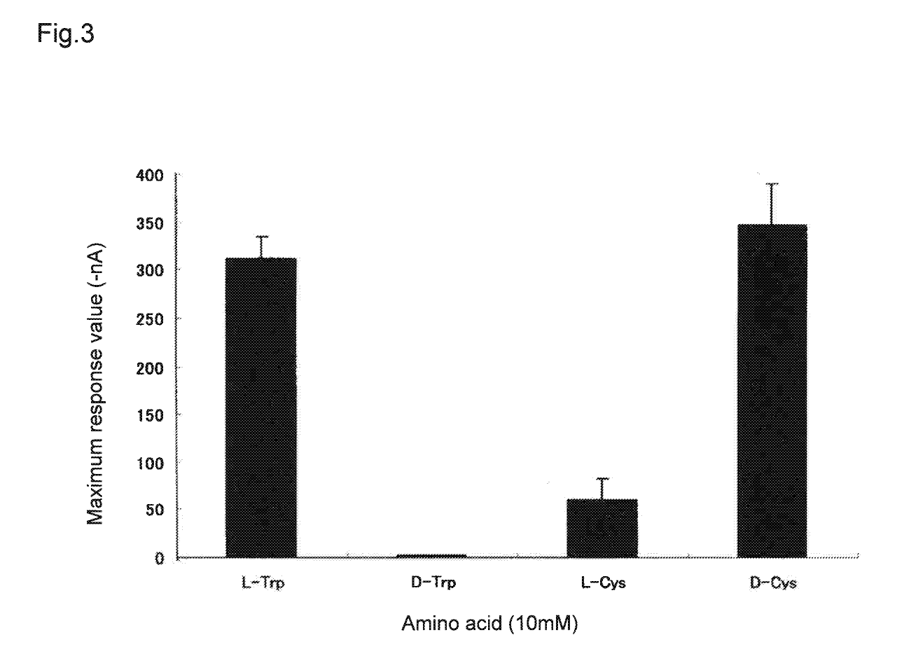

The calcium receptor-activating action of D-cysteine was evaluated by using the method described in Example 13. That is, oocytes injected with cRNA of the calcium receptor or sterilized water were prepared, and membrane potential-clamped at −70 mV by the double electrode potential clamp method. To the potential-clamped oocytes, D-cysteine (10 mM), L-cysteine (10 mM), D-tryptophan (10 mM), or L-tryptophan (10 mM) was added, and Ca ion concentration-dependent Cl response current was measured. FIG. 3 shows the results. The results demonstrated that D-cysteine had a definite calcium receptor-activating action.

### Example 17

### Evaluation of Calcium Receptor-Activating Action of Peptide

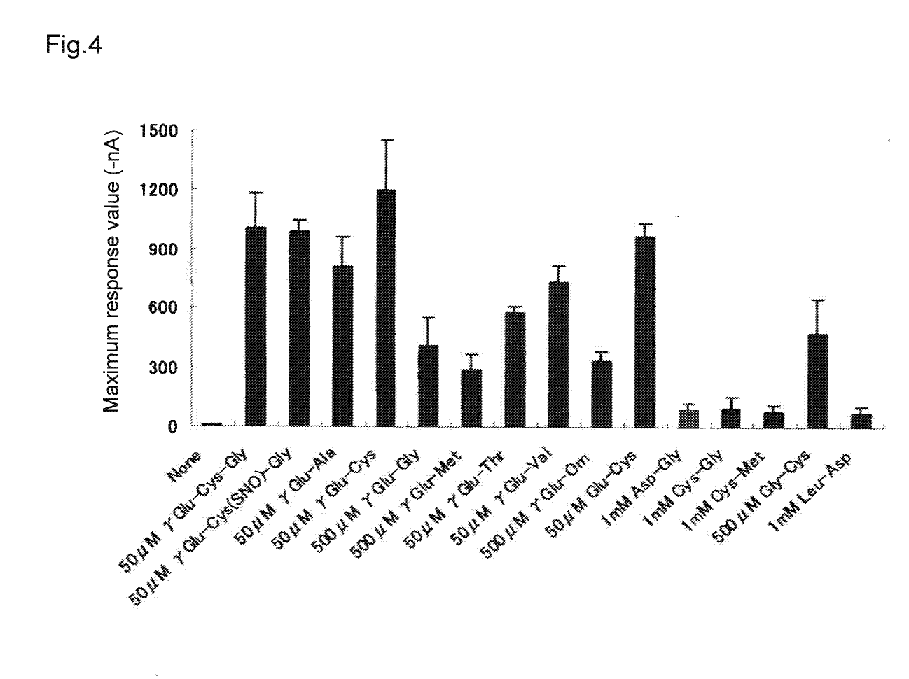

The calcium receptor-activating action of a peptide was evaluated by using the method described in Example 13. That is, oocytes injected with cRNA of the calcium receptor or sterilized water were prepared, and membrane potential-clamped at −70 mV by the double electrode potential clamp method. To the potential-clamped oocytes, γ-Glu-Cys-Gly (50 μM), γ-Glu-Cys(SNO)-Gly (50 μM), γ-Glu-Ala (50 μM), γ-Glu-Gly (500 μM), γ-Glu-Cys (50 μM), γ-Glu-Met (500 μM), γ-Glu-Thr (50 μM), γ-Glu-Val (50 μM), γ-Glu-Orn (500 μM), Asp-Gly (1 mM), Cys-Gly (1 mM), Cys-Met (1 mM), Glu-Cys (50 μM), Gly-Cys (500 μM), or Leu-Asp (1 mM) was added, and Ca ion concentration-dependent Cl response current was measured. FIG. 4 shows the results. The results demonstrated that the above-described peptide had a definite calcium receptor-activating action.

### Example 18

### Evaluation of Calcium Receptor-Activating Action of a Peptide

The calcium receptor-activating action of a peptide was evaluated in the same manner as that of Example 17. Each of the peptides shown in Table 1 was added to potential-clamped oocytes at 1,000 μM, 300 μM, 100 μM, 30 μM, 10 μM, 3 μM, 1 μM, 0.3 μM, and 0.1 μM, and Ca ion concentration-dependent Cl response current was measured. The lowest concentration at which current was detected is shown in Table 1 as the activity. The results revealed that 32 kinds of peptides each had a calcium receptor-activating action.

TABLE 1

| Number | Peptide | Activity | |
|---|---|---|---|
| 1 | γ-Glu-Met(O) | 1,000 | μM |
| 2 | γ-Glu-Val-Val | 1,000 | μM |
| 3 | γ-Glu-Val-Glu | 1,000 | μM |
| 4 | γ-Glu-Val-Lys | 1,000 | μM |
| 5 | γ-Glu-Val-Arg | 1,000 | μM |
| 6 | γ-Glu-Val-Asp | 1,000 | μM |
| 7 | γ-Glu-Val-Met | 1,000 | μM |
| 8 | γ-Glu-Val-Thr | 1,000 | μM |
| 9 | γ-Glu-γ-Glu-Val | 1,000 | μM |
| 10 | γ-Glu-Val-NH₂ | 1,000 | μM |
| 11 | γ-Glu-Val-ol | 1,000 | μM |
| 12 | γ-Glu-Ser | 300 | μM |
| 13 | γ-Glu-Tau | 300 | μM |
| 14 | γ-Glu-Cys(S-Me)(O) | 300 | μM |
| 15 | γ-Glu-Val-His | 100 | μM |
| 16 | γ-Glu-Val-Orn | 100 | μM |
| 17 | γ-Glu-Leu | 100 | μM |
| 18 | γ-Glu-Ile | 100 | μM |
| 19 | γ-Glu-t-Leu | 100 | μM |
| 20 | γ-Glu-Cys(S-allyl)-Gly | 100 | μM |
| 21 | γ-Glu-Val-Asn | 30 | μM |
| 22 | γ-Glu-Gly-Gly | 30 | μM |
| 23 | γ-Glu-Val-Phe | 30 | μM |
| 24 | γ-Glu-Val-Ser | 30 | μM |
| 25 | γ-Glu-Val-Pro | 30 | μM |
| 26 | γ-Glu-Ser-Gly | 30 | μM |
| 27 | γ-Glu-Cys(S-Me) | 30 | μM |
| 28 | γ-Glu-Val-Cys | 10 | μM |
| 29 | γ-Glu-Val-Gln | 10 | μM |
| 30 | γ-Glu-Abu-Gly | 3 | μM |
| 31 | γ-Glu-Cys(S-Me)-Gly | 3 | μM |
| 32 | γ-Glu-Val-Gly | 0.1 | μM |

### Example 19

### Therapeutic Effect on Diarrhea of Peptide which is Able to Activate the Calcium Receptor in an Anticancer Agent-Induced Diarrhea Model

To each of Balb/c mice, an anticancer agent was administered to induce diarrhea, and γ-Glu-Val-Gly (hereinafter, referred to as “γEVG”) was studied for its inhibitory effect on diarrhea. To each of 6-week-old Balb/c mice fed with a low protein nutrient diet (4% casein diet) for 1 week, 5-FU (1 mg/animal/day) was intraperitoneally administered for consecutive 3 days. The diarrhea developed around Day 5 after the third administration of the anticancer agent, and the diarrhea appeared in all cases on Day 7. The presence or absence of diarrhea was determined based on the presence or absence of the stool in the tail head area. As the statistical test, a chi-square (χ²) test for the control group (presence or absence of diarrhea) was employed, and p<0.05 was regarded as significant.

Each administration of 0.01% γEVG was started in free water intake at the same time as the first administration of the anticancer agent, and continued until completion of the experiment.

All (10/10) of the mice exhibited the diarrhea symptom in the control group, while 2 out of 5 mice did not exhibit the diarrhea symptom in the 0.01% γEVG administration group. Those results indicate that γEVG has a significant therapeutic effect on diarrhea in the anticancer agent-induced diarrhea model.

TABLE 2
γEVG administration effect on diarrhea model mice

| | Ratio of |
|---|---|
| | individuals |
| | that developed |
| Medicament | diarrhea |
| Control group | 10/10 |
| 0.01% γEVG | 3/5* |

*p < 0.05 (chi-square test)

### Example 20

### Therapeutic Effect of Peptide which is Able to Activate the Calcium Receptor in a Mouse 5-HTP-Induced Defecation Model

Male ICR mice (5-week-old) were used. To each of the mice, γEVG, which had been dissolved in a 0.5% carboxymethylcellulose aqueous solution, was orally administrated, and after 1 hour, 5-HTP (5-hydroxy tryptophan, 10 mg/kg and 5 ml/kg) was subcutaneously administered. After 30 minutes, the stool form score (0: normal stool or no stool and 1: diarrhea or loose stool) was measured. As a control, a vehicle (0.5% carboxymethylcellulose aqueous solution) free of any medicament was administered to each of the mice.

γEVG was prepared so that the concentrations would be 1% and 0.1% (w/v, hereafter, interpreted with the same meaning).

As the statistical test, a chi-square (χ²) test for the vehicle administration group (presence or absence of diarrhea and loose stool) was employed, and p<0.05 was regarded as significant.

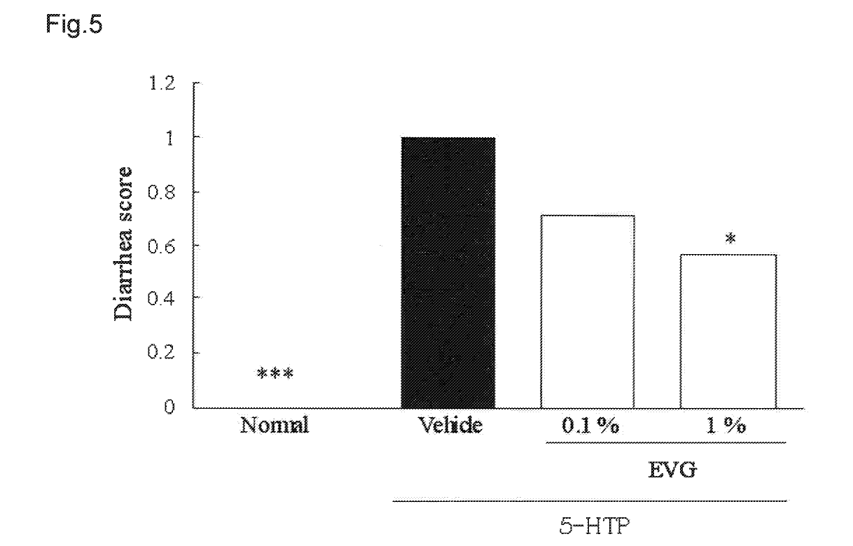

The results are shown in FIG. 5. The stool form score of the vehicle administration group significantly increased compared with that of a healthy group. The administration of 0.1% or 1% γEVG improved the stool form score dose-dependently. In addition, 1% γEVG improved diarrhea significantly.

### Example 21

### Influence of CaSR Activator on Fluid Absorption in Rat Large Intestine Loop Method

<Method>

The cecum and large intestine were extirpated from the abdomen of each of male SD (IGS) rats under pentobarbital anesthesia, and the site 5 cm away from the area just under the cecum was ligated to form a large intestine loop Immediately after the loop had been formed, PGE2 (4 μg/ml/kg, SIGMA) was intraperitoneally administered, and after 30 minutes, a medicament, which had been dissolved in 2 ml of Tyrode's solution (NaCl: 136.9 mM, KCl: 2.7 mM, CaCl₂.2H₂O: 1.8 mM, MgCl₂.6H₂O: 1.04 mM, NaH₂PO₄.2H₂O: 0.04 mM, NaH₂PO₄.2H₂O: 0.04 mM, glucose: 5.55 mM, NaHCO₃: 11.9 mM), was injected into the prepared loop (the medicament solution was adjusted so that the pH would be 6.5 to 7.5). As a control, Tyrode's solution free of any medicament (vehicle) was administered. After 1 hour, the loop weight, the loop weight after solution removal, and the loop area were measured to calculate a solution weight per unit area remaining in the loop.

The remaining solution amount per unit area was calculated with the following equation:

Remaining solution amount per unit area(g/cm²)=(Loop weight−Loop weight after solution removal)/Loop area.

The fluid absorption was evaluated by calculating an inhibitory rate from the following equation:

Inhibitory rate(%)=100−(Remaining solution amount per unit area under drug administration−Remaining solution amount per unit area in base(average))/(Remaining solution amount per unit area under vehicle administration(average)−Remaining solution amount per unit area in base(average))×100.

(Base=case where no stimulation is given (no treatment with PGE2))

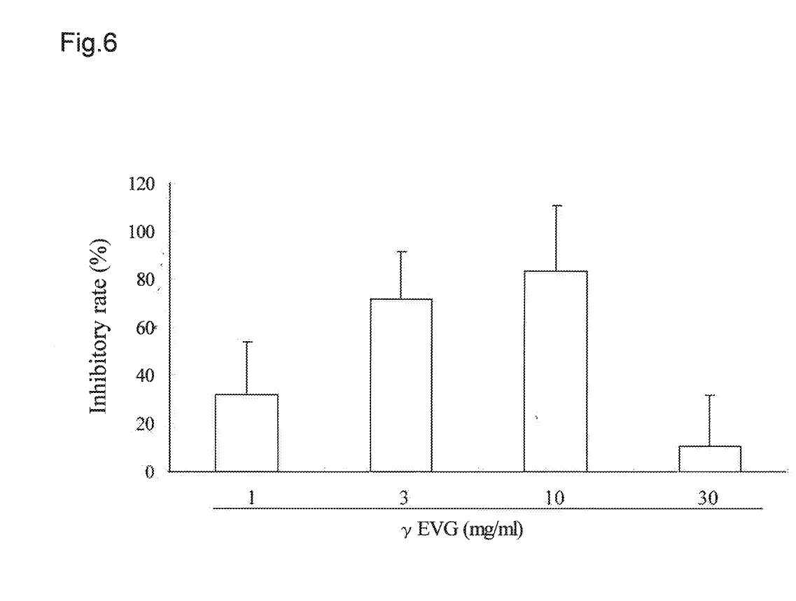

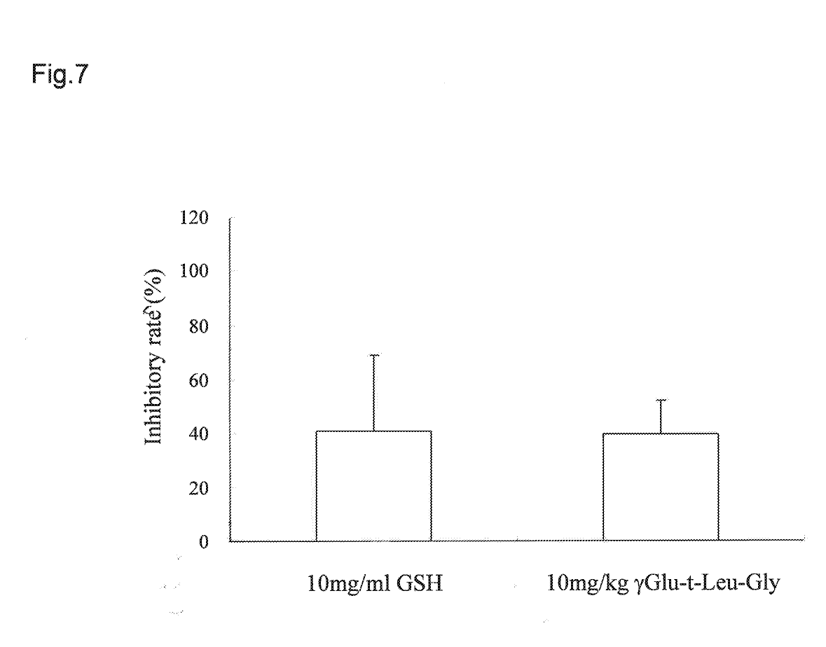

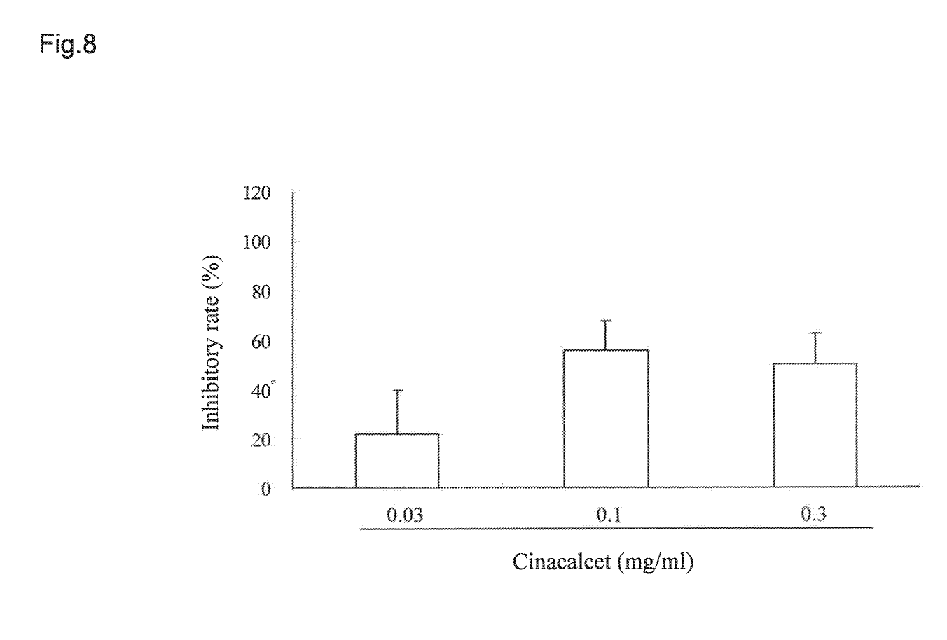

The results are shown in FIGS. 6 to 8. γEVG promoted fluid absorption in a dose-dependent manner. Furthermore, GSH, γ-Glu-t-Leu-Gly, and cinacalcet also promoted fluid absorption. Thus, it was found that fluid absorption promoted by these compound groups provided an inhibitory effect on diarrhea.

### Example 22

### Diarrhea Inhibitory Effect of Calcium Receptor Activator for Anticancer Agent-Induced Diarrhea Model

To each of Balb/c mice, an anticancer agent was administered to induce diarrhea, and a calcium receptor activator (hereinafter, referred to as “CaSR agonist”) was studied for its inhibitory effect on diarrhea. To each of 6-week-old Balb/c mice fed with a low protein nutrient diet (4% casein diet) for 1 week, 5-FU (1 mg/animal/day) was intraperitoneally administered for consecutive 3 days. The diarrhea developed around Day 5 after the third administration of the anticancer agent, and the diarrhea appeared in all cases on Day 7. The presence or absence of diarrhea was determined based on the presence or absence of the stool in the tail head area. As the statistical test, a chi-square (χ²) test for the control group (presence or absence of diarrhea) was employed, and p<0.05 was regarded as significant.

The administration of the CaSR agonist was started in free water intake at the same time as the administration of the low protein nutrient diet, and continued until completion of the experiment.

All (9/9) of the mice exhibited the diarrhea symptom in the control group, while 2 out of 5 mice did not exhibit the diarrhea symptom in both the 0.05% cinacalcet administration group and the 0.5% γ-Glu-Cys-Gly administration group. Those results indicate that the CaSR agonist has a significant inhibitory effect on diarrhea in the anticancer agent-induced diarrhea model.

TABLE 3

| | Ratio of |
|---|---|
| | individuals |
| | that developed |
| Medicament | diarrhea |
| Control group | 9/9 |
| 0.5% cinacalcet | 3/5* |
| 0.5% γ-Glu-Cys-Gly | 3/5* |

*p < 0.05 χ² test for control group (presence or absence of diarrhea)

### Example 23

### Measurement of Activity of CaSR Agonist Peptide

Various peptides synthesized in Example 2 and Examples 6 to 10 were measured for their activities as follows. The cDNA of the human CaSR obtained by the method described in Example 1 of Patent Document WO 2007/055393 A1 was incorporated into an expression vector for animal cells having a CMV promoter, and the gene was introduced into HEK 293 cells which grew up to about 70% of the maximum cell density by using FuGINE 6 (F. Hoffmann-La Roche, Ltd.). After the culture for 4 to 48 hours, the cells were seeded into a 96-well plate in an amount of 0.6 to 1.0×10⁵ cells/well, and cultured for 1 day. After that, the cells were stained with a Calcium 3 assay kit. The presence or absence of the activity of the CaSR agonist peptide was confirmed by a calcium imaging method using FLEXSTATION (MDS Inc., instruction manual). In addition, the concentration that provides 50% of the maximum activity was determined as EC50 from a dose characteristic curve.

TABLE 4

| Compounds | EC50, μM |
|---|---|
| γ-Glu-Val-Gly | 0.023-0.055 |
| γ-Glu-Cle-Gly | 0.5-1.5 |
| γ-Glu-Aib-Gly | 0.349-1.167 |
| γ-Glu-t-Leu-Gly | 0.043-0.077 |
| γ-Glu-Pen-Gly | 0.159-0.233 |
| γ-Glu-Val-GlyA | 0.14 |
| γ-Glu-t-Leu-GlyA | 0.05 |
| γ-Glu-Abu-GlyA | 0.065-0.079 |
| γ-Glu-Val-LacA | 0.057 |
| γ-Glu-t-Leu-LacA | 0.038 |
| γ-Glu-Abu-LacA | 0.102 |

*The experimental value including only one numerical value indicates a value with n = 1, and the other experimental values indicate a range of values with n = 2 to 3.

### INDUSTRIAL APPLICABILITY

The present invention provides a prophylactic or therapeutic agent for diarrhea, which is highly safe to the living body.

While the invention has been described in detail with reference to exemplary embodiments thereof, it will be apparent to one skilled in the art that various changes can be made, and equivalents employed, without departing from the scope of the invention. Each of the aforementioned documents is incorporated by reference herein in its entirety.

What is claimed is:

1. A prophylactic or therapeutic agent for treating diarrhea comprising a compound which is able to activate a calcium receptor.

2. The prophylactic or therapeutic agent according to claim 1, wherein the compound is selected from the group consisting of a peptide, a peptide derivative, cinacalcet, a compound having the structure of formula (1), and a compound having the structure of formula (2).

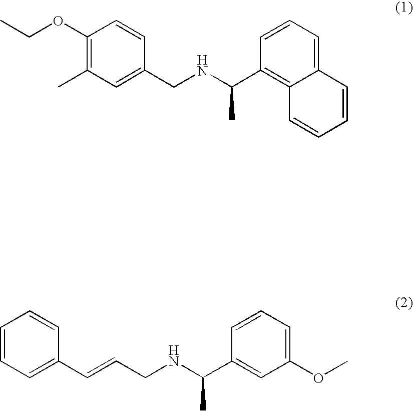

3. The prophylactic or therapeutic agent according to claim 2, wherein the peptide is selected from the group consisting of γ-Glu-X-Gly, γ-Glu-Val-Y, γ-Glu-Ala, γ-Glu-Gly, γ-Glu-Cys, γ-Glu-Met, γ-Glu-Thr, γ-Glu-Val, γ-Glu-Orn, Asp-Gly, Cys-Gly, Cys-Met, Glu-Cys, Gly-Cys, Leu-Asp, γ-Glu-Met(O), γ-Glu-γ-Glu-Val, γ-Glu-Val-NH₂, γ-Glu-Val-ol, γ-Glu-Ser, γ-Glu-Tau, γ-Glu-Cys(S-Me)(O), γ-Glu-Leu, γ-Glu-Ile, γ-Glu-t-Leu, γ-Glu-Cys(S-Me), and combinations thereof, wherein X and Y are an amino acid or an amino acid derivative.

4. The prophylactic or therapeutic agent according to claim 3, wherein X is selected from the group consisting of Cys(SNO), Cys(S-allyl), Gly, Cys(S-Me), Cys, Abu, t-Leu, Cle, Aib, Pen, and Ser; and Y is selected from the group consisting of Gly, Val, Glu, Lys, Phe, Ser, Pro, Arg, Asp, Met, Thr, His, Orn, Asn, Cys, and Gln.

5. The prophylactic or therapeutic agent according to claim 3, wherein the peptide is selected from the group consisting of γ-Glu-Val-Gly and γ-Glu-t-Leu-Gly.

6. The prophylactic or therapeutic agent according to claim 2, wherein the peptide derivative has a structure γ-Glu-X—OCH(Z)CO₂H, wherein X is an amino acid or an amino acid derivative, and Z is H or CH₃.

7. A compound having the structure γ-Glu-X—OCH(Z)CO₂H, wherein X is an amino acid or an amino acid derivative, and Z is H or CH₃.

8. The compound according to claim 7, wherein X is t-Leu or Abu.

9. A compound having the formula γ-Glu-t-Leu-Gly.
